(19) United States
(12) Patent Application Publication
(10) Pub. No.: US 2023/0190201 A1
(43) Pub. Date: Jun. 22, 2023

(54) TECHNIQUES FOR MULTIPLE WEARABLE DEVICES

(71) Applicant: Oura Health Oy, Oulu (FI)

(72) Inventors: Alec Singleton, Toronto (CA); Steve Kent, San Francisco, CA (US); Mika Erkkilä, Oulu (FI); Heli Tuulia Koskimäki, Oulu (FI); Michael Chapp, Lafayette, CA (US)

(21) Appl. No.: 18/074,343

(22) Filed: Dec. 2, 2022

Related U.S. Application Data
(60) Provisional application No. 63/292,937, filed on Dec. 22, 2021.

(51) Int. Cl. A61B 5/00; A61B 5/11; A61B 5/0205

(52) U.S. Cl. CPC A61B 5/7221; A61B 5/7425; A61B 5/1118; A61B 5/0205; A61B 5/6826; A61B 5/7267; A61B 5/0002; A61B 5/026

(57) ABSTRACT

Methods, systems, and devices for multiple wearable devices are described. A method may include receiving first physiological data associated with a user from a first wearable device worn at a first position on the user and second physiological data associated with the user from a second wearable device worn at a second position on the user. This method may include determining one or more physiological characteristics associated with the user based on a comparison of the first physiological data and the second physiological data and displaying an indication of the one or more physiological characteristics on a graphical user interface (GUI) of a user device.

### CROSS REFERENCE

The present Application for Patent claims the benefit of U.S. Provisional Patent Application No. 63/292,937 by SINGLETON et al., entitled “TECHNIQUES FOR MULTIPLE WEARABLE DEVICES,” filed Dec. 22, 2021, assigned to the assignee hereof, and expressly incorporated by reference herein.

### FIELD OF TECHNOLOGY

The following relates to wearable devices and data processing, including techniques for multiple wearable devices.

### BACKGROUND

Some wearable devices may be configured to collect data from users associated with motion data, temperature data, heart rate data, photoplethysmography (PPG) data, and the like. Some users have a desire for more insights regarding their physiological data and overall health.

### BRIEF DESCRIPTION OF THE DRAWINGS

FIG. 1 illustrates an example of a system that supports techniques for multiple wearable devices in accordance with aspects of the present disclosure.

FIG. 2 illustrates an example of a system that supports techniques for multiple wearable devices in accordance with aspects of the present disclosure.

FIG. 3 illustrates an example of a system that supports techniques for multiple wearable devices in accordance with aspects of the present disclosure.

FIG. 4 illustrates an example of a graphical user interface (GUI) that supports techniques for multiple wearable devices in accordance with aspects of the present disclosure.

FIG. 5 shows a block diagram of an apparatus that supports techniques for multiple wearable devices in accordance with aspects of the present disclosure.

FIG. 6 shows a block diagram of a wearable application that supports techniques for multiple wearable devices in accordance with aspects of the present disclosure.

FIG. 7 shows a diagram of a system including a device that supports techniques for multiple wearable devices in accordance with aspects of the present disclosure.

FIGS. 8 through 10 show flowcharts illustrating methods that support techniques for multiple wearable devices in accordance with aspects of the present disclosure.

### DETAILED DESCRIPTION

A user may use a device (e.g., a wearable device) to determine physiological measurements of the user, such as motion data, temperature data, heart rate data, photoplethysmography (PPG) data, and the like. Physiological data collected via a wearable device may be used to gain insights into the user's sleeping patterns and overall health. However, in some cases, data collected via a wearable device may not accurately represent a user's physiological parameters. For example, liquids, sweat, dirt, and other substances between a wearable device and a user's skin may negatively affect the accuracy of physiological data collected by the wearable device. By way of another example, in cases where a user uses a single wearable device located on their wrist, the wearable device may be unable to accurately detect and track workouts that are performed primarily with the legs, such as cycling workouts.

Additionally, some physiological parameters may be more effectively and accurately measured at some locations of the body as compared to other locations of the body. For example, body temperature readings may be more effectively collected via wearable devices worn around the user's chest or torso as compared to the user's extremities (e.g., arms, hands, legs, feet). Conversely, motion and accelerometer data may be more accurately and reliably tracked via wearable devices worn on the user's arms or legs as opposed to wearable devices worn on the user's torso. In this regard, a wearable device worn at a single location on a user's body may be unable to accurately and reliably collect physiological data for multiple physiological parameters.

Accordingly, in order to provide more accurate and reliable physiological data, and to provide more insights into a user's overall health, aspects of the present disclosure support techniques for multiple wearable devices. In particular, aspects of the present disclosure relate to techniques for collecting physiological data from multiple wearable devices to improve measurement accuracy, decrease battery consumption, and provide additional insight into a user's health. In some cases, a user may be associated with multiple wearable devices that may be worn on different parts of the body, such as a ring wearable device located on a finger and a chest-worn wearable device. Accordingly, each wearable device may collect physiological data associated with the user. The physiological data collected from the multiple wearable devices may be compared to determine one or more physiological characteristics associated with the user. In some aspects, a graphical user interface (GUI) of a user device may display an indication of the one or more physiological characteristics determined based on the physiological data collected via one or more wearable devices (e.g., two wearable devices, three wearable devices, etc.).

In some cases, quality metrics may be associated with the physiological data collected by the multiple wearable devices. For example, a first quality metric may be associated with first physiological data collected from a first wearable device and a second quality metric may be associated with second physiological data collected from a second wearable device. A comparison of the quality metrics may be used to determine modification of one or more operational parameters associated with a wearable device. For example, in cases where the first quality metric is greater than the second quality metric (indicating that the first wearable device is collecting higher-quality data), aspects of the present disclosure may modify an operational parameter associated with the second wearable device. In some cases, modifying one or more operational parameters may include transitioning one or more wearable devices from an active state to an inactive state (e.g., deactivating the second wearable device), modifying a current provided to one or more sensors of the wearable device (e.g., increasing a current or voltage provided to the second wearable device), or both.

In some aspects, techniques described herein may utilize multiple wearable devices to provide additional insights into a user's physiological data and overall health that may not be determined using a single wearable device. For example, motion data collected via multiple wearable devices may be compared in order to more accurately classify physical activities. For instance, in cases where a wearable device worn on the user's finger exhibits low motion, but a wearable device worn on the user's leg exhibits higher motion, it may be more likely that the user is performing a cycling workout as compared to a running workout. Moreover, in some aspects, physiological data collected via multiple wearable devices on opposite sides of the body (e.g., wearable devices worn on both the left and right hands) may provide insights regarding several physiological conditions, including Alzheimer's, Parkinson's, strokes, and the like.

Aspects of the disclosure are initially described in the context of systems supporting physiological data collection from users via wearable devices. Aspects are then described with reference to a GUI. Aspects of the disclosure are further illustrated by and described with reference to apparatus diagrams, system diagrams, and flowcharts that relate to techniques for multiple wearable devices.

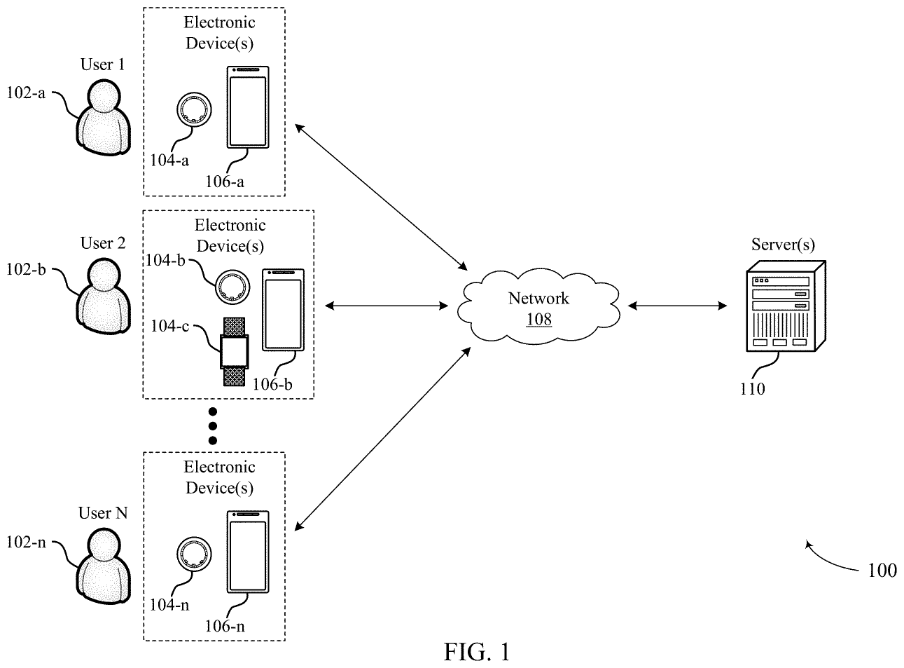

FIG. 1 illustrates an example of a system 100 that supports techniques for multiple wearable devices in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during that a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for multiple wearable devices. In particular, aspects of the present disclosure relate to techniques for collecting physiological data from multiple wearable devices 104 to improve measurement accuracy, decrease battery consumption, and provide additional insight into a user's 102 health. In some cases, a user 102 may be associated with multiple wearable devices 104 positioned on different parts of the body. The multiple wearable devices 104 may collect physiological data from the multiple positions. For example, a user 102-b may wear a ring 104-b on a finger (e.g., a first position) and a watch (or other wrist-worn wearable) 104-c on a wrist (e.g., a second position). As such, the ring 104-b may collect physiological data from the first position and the watch 104-c may collect physiological data from the second position. In some cases, the system 100 may determine one or more physiological characteristics based on a comparison of the physiological data collected at the first position and the physiological data collected at the second position. Additionally, the system 100 may display the one or more physiological characteristics via a GUI on a user device 106-b.

In some cases, the ring 104-b and the watch 104-c may be configured to communicate directly, through the user device 106-b, directly with the network 108, or any combination thereof (e.g., producing a mesh network). In some cases, each wearable device 104 may communicate collected physiological data to a user device 106-b. For example, the ring 104-b may communicate the physiological data collected from the first position to the user device 106-b and the watch 104-c may communicate the physiological data collected at the second position to the user device 106-b. Additionally or alternatively, a subset of the wearable devices 104 may aggregate the physiological data collected via the multiple wearable devices 104 and may communicate the aggregated physiological data to the user device 106-b. For example, the ring 104-b may communicate the physiological data collected at the first position to the watch 104-c, the watch 104-c may aggregate the physiological data collected at the first position with the physiological data collected at the second position (e.g., collected by the watch 104-c), and the watch 104-c may communicate the aggregated physiological data to the user device 106-b.

In some cases, the system 100 (e.g., user devices 106, servers 110) may determine quality metrics associated with the physiological data collected by the multiple wearable devices 104. As it is used herein, the term “quality metric” may refer to some metric indicative of a relative accuracy, quality, or reliability of collected data. For example, a first quality metric may be associated with first physiological data collected from a first wearable device 104, such as the ring 104-b, and a second quality metric may be associated with second physiological data collected from a second wearable device 104, such as the watch 104-c. A comparison of the quality metrics may be used to modify one or more operational parameters associated with a wearable device 104. For example, in cases where a first quality metric associated with physiological data collected by the ring 104-b exceeds a second quality metric associated with the physiological data collected by the watch 104-c, the system 100 may modify one or more operational parameters associated with the watch 104-c. For instance, the system 100 (e.g., user device 106) may deactivate the watch 104-c (e.g., one or more sensors of the watch 104-c) to conserve battery power (e.g., transition the watch 104-c from an active to an inactive state), or may increase a current or voltage provided to sensors of the watch 104-c in an attempt to improve the quality of physiological data collected via the watch 104-c.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

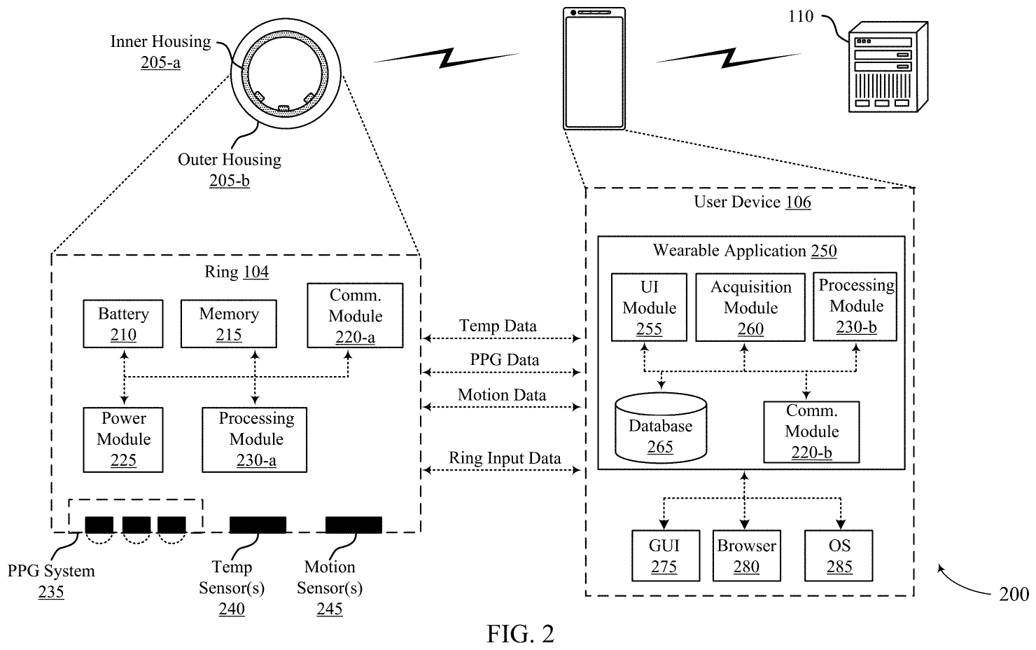

FIG. 2 illustrates an example of a system 200 that supports techniques for multiple wearable devices in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmography (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamp, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for multiple wearable devices. In particular, aspects of the present disclosure relate to techniques for collecting physiological data from multiple wearable devices 104 to improve measurement accuracy, decrease battery consumption, and provide additional insight into a user's 102 health. In some cases, a user 102 may be associated with multiple wearable devices 104 positioned on different parts of the body. The multiple wearable devices 104 may collect physiological data from the multiple positions. For example, each of the multiple wearable devices 104 may collect physiological data from one or more of the PGG system 235, temperature sensors 240, or motion sensors 245. In some cases, the system 200 may determine one or more physiological characteristics based on a comparison of the physiological data collected by a wearable device 104 at a first position and the physiological data collected by a wearable device 104 at a second position. Additionally, the server 110 of system 200 may cause the GUI 275 of the user device 106 (e.g., mobile device) to display an indication of the physiological characteristics via the wearable application 250.

In some cases, the multiple wearable devices 104 may be configured to communicate directly, through the user device 106, directly with a network 108 (as described in FIG. 1), or any combination thereof (e.g., producing a mesh network). In some cases, each wearable device 104 may communicate collected physiological data to a user device 106. For example, the ring 104 may communicate the physiological data collected from a first position to the user device 106 via the communication module 220-a and the communication module 220-b. Additionally or alternatively, a subset of the wearable devices 104 may aggregate the physiological data collected via the multiple wearable devices 104 and may communicate the aggregated physiological data to the user device 106. For example, a wearable device 104 may collect physiological data from a second position and communicate the physiological data to the ring 104 via a communication module 220. The ring 104 may aggregate the additional physiological data with the physiological data collected by the ring 104 at the first position and communicate the aggregated data to the user device 106 via the communication module 220-a and the communication module 220-b.

In some cases, the system 200 (e.g., wearable devices 104, user devices 106, servers 110) may determine quality metrics associated with the physiological data collected by the multiple wearable devices 104. For example, the system 200 may determine a first quality metric associated with first physiological data collected from a first wearable device 104, such as the ring 104, and a second quality metric associated with second physiological data collected from a second wearable device 104. A comparison of the quality metrics may be used to selectively modify one or more operational parameters associated with a wearable device 104 (e.g., one or more sensors or components of the wearable device 104). Operational parameters of wearable devices 104 that may be modified may include, but are not limited to, operational states (e.g., active state, inactive state, reduced-power state), currents/voltages applied to components of the wearable devices 104, physiological measurements performed by the respective wearable devices 104, and the like.

For example, the system 200 may determine a first quality metric associated with physiological data collected by the ring 104 and a second quality metric associated with the physiological data collected by a second wearable device 104. The system 200 may determine that the second quality metric may be greater than the first quality metric (e.g., the second wearable device 104 is collecting higher-quality data). In this example, the system 200 may modify an operational parameter associated with the ring 104, the second wearable device 104, or both, based on the comparison of the quality metrics. In some cases, modifying one or more operational parameters may include transitioning one or more wearable devices 104 from an active state to an inactive state, modifying a current provided to one or more sensors of the one or more wearable devices 104, or both. For example, the system 200 may modify the operational parameters of the ring 104 such that the power module 225 increases or decreases the current provided to one or more of the PPG system 235, temperature sensors 240, or motion sensors 245 based on the comparison of the quality metrics. Modifying the operation parameters may result in decreased battery consumption and improved life of the battery 210.

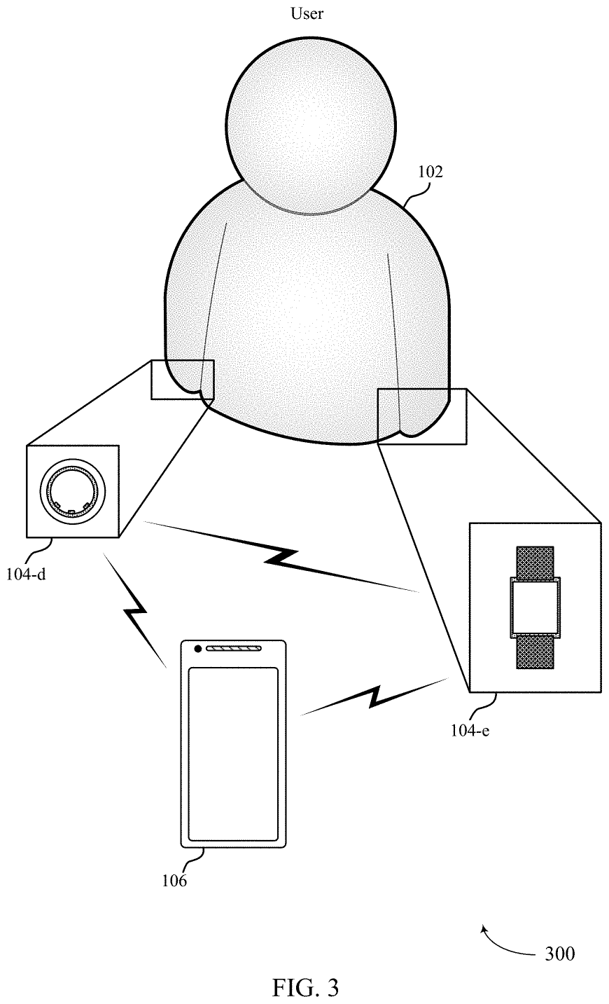

FIG. 3 illustrates an example of a system 300 that supports techniques for multiple wearable devices in accordance with aspects of the present disclosure. The system 300 may implement, or be implemented by, the system 100 and the system 200. In particular, the system 300 illustrates an example of a ring 104-d (e.g., a wearable device 104), a watch 104-e (e.g., a wearable device 104), a user device 106, and a user 102, as described with reference to FIG. 1.

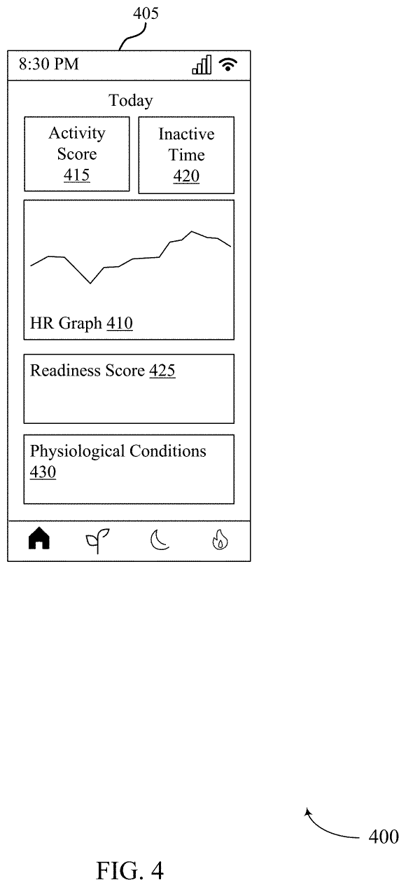

In some cases, the user 102 may wear the ring 104-d on a finger (e.g., at a first position) and the watch 104-e on a wrist (e.g., at a second position). The ring 104-d may collect first physiological data from the first position and the watch 104-e may collect second physiological data from the second position. In some cases, the system 300 may be configured to collect and evaluate the physiological data collected by each of the sensors on the wearable devices 104. As such, the system 300 may receive the first physiological data associated with the user 102 from the ring 104-d worn at the first position, as well as the second physiological data associated with the user 102 from the watch 104-e worn at the second position. The system 300 may compare the first physiological data and the second physiological data to determine one or more physiological characteristics associated with the user 102. Additionally, the system 300 may display an indication of the one or more physiological characteristics via a GUI on user device 106. Presentation of the physiological characteristics will be further shown and described with reference to FIG. 4.

In some cases, the wearable devices 104 may be configured to communicate directly, through the user device 106, directly with a network 108 (as described in FIG. 1), or any combination thereof (e.g., producing a mesh network). In some cases, each wearable device 104 may communicate collected physiological data to a user device 106. For example, the ring 104-d and the watch 104-e may each communicate the physiological data collected at the respective positions to the user device 106. Additionally or alternatively, a subset of the wearable devices 104 may aggregate the physiological data collected via the multiple wearable devices 104 and may communicate the aggregated physiological data to the user device 106. For example, the ring 104-d may communicate the first physiological data to the watch 104-e and the watch 104-e may aggregate the first physiological data with the second physiological data (e.g., collected by the watch 104-e). In this example, the watch 104-e may communicate the aggregated physiological data to the user device 106.

Collecting and comparing physiological data from multiple wearable devices 104 may enable the system 300 to measure different physiological parameters using different wearable devices 104 located at different positions on the user 102. That is, the system 300 may optimize physiological data collection such that a wearable device 104 may collect a subset of physiological data, where the subset of physiological data is associated with a strong physiological data collection accuracy. For example, the ring device 104-d may collect motion data at the finger (e.g., where the motion data may be more accurate), while a wearable device 104 at the chest may collect temperature data (e.g., where the temperature data may be more accurate due to the sensor proximity to the core of the user 102).

Comparing physiological data collected by the multiple wearable devices 104 may provide insight into the user's 102 health. For example, the one or more physiological characteristics determined based on the comparison of the physiological data collected by the multiple wearable devices 104 may include a metric associated with blood circulation of the user 102, one or more risk metrics associated with one or more medical conditions, or both.

In some cases, the one or more medical conditions may include Parkinson's, Alzheimer's, stroke, or any combination thereof. In particular, some medical conditions (e.g., Parkinson's, Alzheimer's) may manifest differently, or at different rates, on one side of the body as compared to the other side of the body. For instance, oftentimes symptoms of a stroke only (or primarily) manifest on one side of the body. As such, collecting physiological data via both wearable devices 104-d and 104-e (e.g., wearable ring devices 104 on each hand) may provide more insight into such medical conditions, that may lead to earlier diagnosis and treatment. For instance, studies have found that Parkinson's often begins with symptoms that begin mostly (or exclusively) one side of the body. Accordingly, by detecting baseline changes in physiological data happening on one side of the body (but not the other), aspects of the present disclosure may facilitate early Parkinson's diagnosis. Moreover, as physiological data is collected, the system 300 may detect that the changes attributable to Parkinson's may be shifting to the rest of the body, that may provide insight as to how the disease is developing and/or becoming more severe. Furthermore, studies have shown that the difference between physiological data collected on a user's left and right sides may provide more insight into certain medical conditions (e.g., stress, Parkinson's, Alzheimer's) than the actual physiological values collected from either respective side.

For example, at a first time, the system 300 may use physiological data collected at a first location to determine a metric associated with a medical condition (e.g., Parkinson's). Further, physiological data collected at a second location at the first time may not result in determination of the metric associated with the medical condition (e.g., the symptoms may only be present at the first position on the user 102). At a second time, the system 300 may use physiological data collected at the first location and the second location to determine the metric associated with the medical condition. That is, the system 300 may identify that the medical condition has progressed. In another example, the ring 104-d may collect physiological data at the first position based on arterial blood flow. Additionally or alternatively, the watch 104-e may collect physiological data at the second position based on arterial blood flow. The system 300 may use the physiological data collected at the first position, the physiological data collected at the second position, or both, to determine metrics associated with blood circulation of the user.

In some cases, the system 300 may associate the one or more physiological characteristics from the user 102 with data from additional sources to identify relationships and trends and improve health-related insights and messaging. For example, the system 300 may identify that physiological data collected from the right side of a user 102 differs from physiological data collected from the left side of the user. The system 300 may associate the difference in physiological data with additional sources (e.g., studies) to identify one or more physiological characteristics associated with stress or other medical conditions, such as Parkinson's and Alzheimer's.

In some cases, the system 300 may collect physiological data from the multiple wearable devices 104 to improve activity classification and detection. For example, the physiological data collected from the multiple wearable devices 104 may include motion data and the system 300 may identify the user 102 is engaged in an activity based on the motion data. Additionally, the system 300 may classify the physical activity into a physical activity type from multiple candidate physical activity types based on a comparison of the motion data. For example, the ring 104-d may collect first motion data (e.g., a periodic accelerometer reading) at a first position and the watch 104-e may collect second motion data at a second position. The system 300 may identify that the first motion data and the second motion data are similar and may classify the physical activity type based on the identification (e.g., classify the activity as running). In another example, the ring 104-d may collect first motion data at the first position that differs from second motion data collected by a wearable device 104 located on the leg (e.g., a second position) of the user 102 (e.g., higher accelerometer readings from the sensors on the wearable device 104 located on the leg of the user 102). In this example, the system 300 may compare the first motion data and the second motion data and may classify the physical activity type based on the identification (e.g., classify the activity as biking).

In some cases, one or more wearable devices 104 may be activated and deactivated (e.g., transition from an active state to an inactive state) based on physiological data collection quality. For example, the system 300 may determine a first quality metric for the physiological data collected by the ring 104-d and a second quality metric for the physiological data collected by the watch 104-e. The system 300 may identify that the first quality metric is greater, or is associated with a higher accuracy, than the second quality metric. The system 300 may modify the operational parameters of the watch 104-e based on the determination that the second quality metric is lower than the first quality metric. For instance, the system 300 may modify the operational parameters by changing the watch 104-e (e.g., or one or more components of the watch 104-e) from an active state to an inactive state due to the lower quality metrics, thereby reducing power consumption and improving battery life at the watch 104-e. By way of another example, the system 300 may increase a current or voltage provided to one or more sensors of the watch 104-e in an attempt to increase the quality of physiological measurements performed by the watch 104-e.

In some aspects, physiological data collected via the ring 104-d, the watch 104-e, or both, may be used to calculate/modify one or more scores for the user 102, such as the user's Sleep Score, Readiness Score, Activity Score, or any combination thereof. Although the system 300 is shown and described as including a ring 104-d, a watch 104-e, and a user device 106, it is understood that any quantity and combination of wearable devices 104 may support the techniques described herein.

While FIG. 3 is shown and described as including a user 102 wearing a wearable ring device 104-d and a wearable watch device 104-e, this is not to be regarded as a limitation of the present disclosure, unless noted otherwise herein. In this regard, aspects of the present disclosure may be implemented in the context of any type or quantity of wearable devices 104, that may be worn on/at any part of a user's body or clothing. For example, aspects of the present disclosure may be implemented in the context of chest-worn devices (e.g., chest straps), wearable devices worn on the user's legs (e.g., button sensors on legs/pants used for gait analysis, sensors on legs/feet/toes), wearable devices worn around the neck (e.g., wearable necklace devices), wearable earring devices, wearable headbands or other headwear devices, and the like.

For example, in some implementations, the system 300 may include one or more wearable devices 104 worn on the legs of the user 102. Leg-worn sensors may be used by the system 300 to analyze the user's gait, to diagnose periodic limb movement disorder, and the like. Moreover, one or more leg-worn wearable devices 104 may be used to improve activity detection and classification.

FIG. 4 illustrates an example of a GUI 400 that supports techniques for multiple wearable devices in accordance with aspects of the present disclosure. The GUI 400 may implement, or be implemented by, aspects of the system 100, system 200, system 300, or any combination thereof. For example, the GUI 400 may include an example of the GUI 275 included within the user device 106 illustrated in FIG. 2.

The GUI 400 illustrates an application page 405 that may be displayed to the user via the GUI 400 (e.g., GUI 275 illustrated in FIG. 2). The server 110 of system 200 may cause the GUI 400 of the user device 106 (e.g., mobile device) to display an indication of the physiological characteristics (e.g., via the application page 405). Accordingly, upon determining physiological characteristics (e.g., as described with reference to FIG. 3), the user may be presented with the application page 405 upon opening the wearable application 250. For example, the application page 405 may display one or more messages or other indications associated with one or more physiological conditions 430 determined based on physiological data collected via one or more wearable devices 104.

As shown in FIG. 4, the application page 405 may display a heart rate graph 410 (e.g., a physiological characteristic). The heart rate graph 410 may include a visual representation of how the user's heart rate reacted to different events and activities (e.g., exercise, sleep, rest, etc.). Additionally, in some implementations, the application page 405 may display one or more scores (e.g., a Sleep Score, an Activity Score 415, an inactive time 420, a Readiness Score 425) for the user for the respective day (e.g., respective sleep day), where the one or more scores may be based on the physiological data (e.g., the first physiological data and the second physiological data). By way of another example, the physiological data may be used to update at least a subset of the factors for the Readiness Score 415 (e.g., subset of sleep, sleep balance, HRV balance, recovery index, activity, activity balance).

The server 110 of system 200 may cause the GUI 400 of the user device to display a message on the application page 405, associated with the identified physiological characteristics. The user device may display recommendations and/or information associated with the physiological characteristic data via a message. In some implementations, the user device 106 and/or servers 110 may generate alerts (e.g., messages, insights) associated with the physiological characteristic that may be displayed to the user via the GUI 400 (e.g., the application pages 405 or some other application page). In particular, the messages generated and displayed to the user via the GUI 400 may be associated with one or more characteristics (e.g., time of day, duration, range) of the physiological characteristic. For example, the message may alert the user to breathe, take a moment to relax, etc., based on the user's heart rate. In some cases, the message may display a recommendation of how to adjust their lifestyle to achieve a particular physiological characteristic. In this regard, the system may be configured to display messages or insights to the user in order to facilitate effective, healthy patterns for the user.

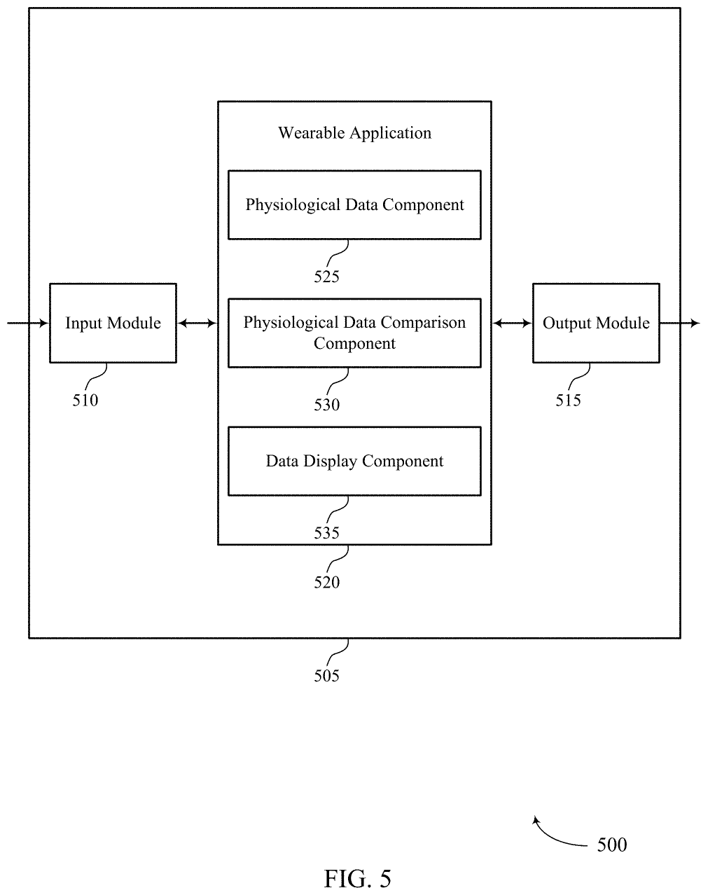

FIG. 5 shows a block diagram 500 of a device 505 that supports techniques for multiple wearable devices in accordance with aspects of the present disclosure. The device 505 may include an input module 510, an output module 515, and a wearable application 520. The device 505 may also include a processor. Each of these components may be in communication with one another (e.g., via one or more buses).

The input module 510 may provide a means for receiving information such as packets, user data, control information, or any combination thereof associated with various information channels (e.g., control channels, data channels, information channels related to illness detection techniques). Information may be passed on to other components of the device 505. The input module 510 may utilize a single antenna or a set of multiple antennas.

The output module 515 may provide a means for transmitting signals generated by other components of the device 505. For example, the output module 515 may transmit information such as packets, user data, control information, or any combination thereof associated with various information channels (e.g., control channels, data channels, information channels related to illness detection techniques). In some examples, the output module 515 may be co-located with the input module 510 in a transceiver module. The output module 515 may utilize a single antenna or a set of multiple antennas.

For example, the wearable application 520 may include a physiological data component 525, a physiological data comparison component 530, a data display component 535, or any combination thereof. In some examples, the wearable application 520, or various components thereof, may be configured to perform various operations (e.g., receiving, monitoring, transmitting) using or otherwise in cooperation with the input module 510, the output module 515, or both. For example, the wearable application 520 may receive information from the input module 510, send information to the output module 515, or be integrated in combination with the input module 510, the output module 515, or both to receive information, transmit information, or perform various other operations as described herein.

The wearable application 520 may support collecting physiological data from a plurality of wearable devices in accordance with examples as disclosed herein. The physiological data component 525 may be configured as or otherwise support a means for receiving first physiological data associated with a user from a first wearable device worn at a first position on the user. The physiological data component 525 may be configured as or otherwise support a means for receiving second physiological data associated with the user from a second wearable device worn at a second position on the user different from the first position. The physiological data comparison component 530 may be configured as or otherwise support a means for determining one or more physiological characteristics associated with the user based at least in part on a comparison of the first physiological data and the second physiological data. The data display component 535 may be configured as or otherwise support a means for causing a graphical user interface of a user device to display an indication of the one or more physiological characteristics.

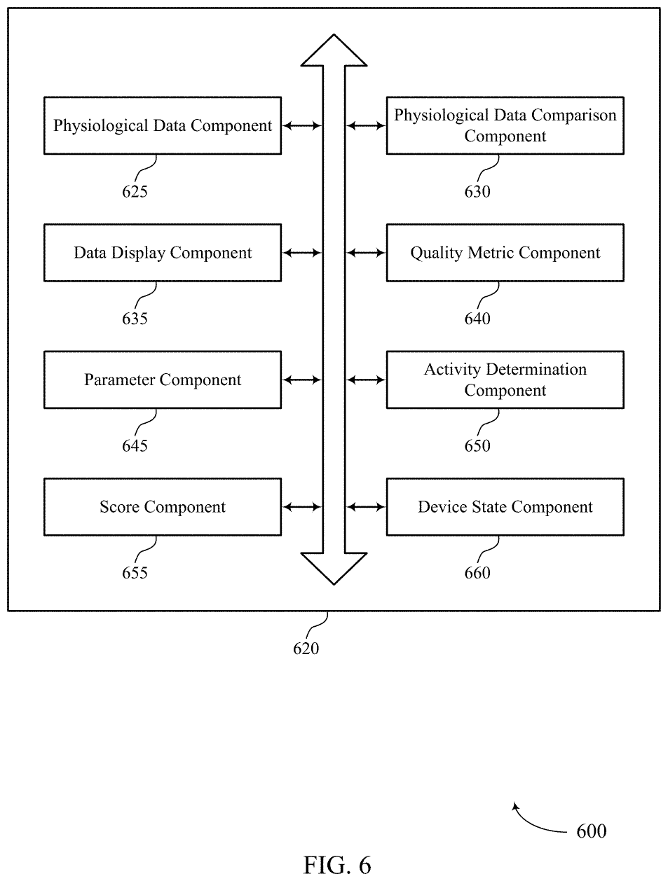

FIG. 6 shows a block diagram 600 of a wearable application 620 that supports techniques for multiple wearable devices in accordance with aspects of the present disclosure. The wearable application 620 may be an example of aspects of a wearable application or a wearable application 520, or both, as described herein. The wearable application 620, or various components thereof, may be an example of means for performing various aspects of techniques for multiple wearable devices as described herein. For example, the wearable application 620 may include a physiological data component 625, a physiological data comparison component 630, a data display component 635, a quality metric component 640, a parameter component 645, an activity determination component 650, a score component 655, a device state component 660, or any combination thereof. Each of these components may communicate, directly or indirectly, with one another (e.g., via one or more buses).

The wearable application 620 may support collecting physiological data from a plurality of wearable devices in accordance with examples as disclosed herein. The physiological data component 625 may be configured as or otherwise support a means for receiving first physiological data associated with a user from a first wearable device worn at a first position on the user. In some examples, the physiological data component 625 may be configured as or otherwise support a means for receiving second physiological data associated with the user from a second wearable device worn at a second position on the user different from the first position. The physiological data comparison component 630 may be configured as or otherwise support a means for determining one or more physiological characteristics associated with the user based at least in part on a comparison of the first physiological data and the second physiological data. The data display component 635 may be configured as or otherwise support a means for causing a graphical user interface of a user device to display an indication of the one or more physiological characteristics.

In some examples, the quality metric component 640 may be configured as or otherwise support a means for identifying that a first quality metric associated with the first physiological data is greater than a second quality metric associated with the second physiological data. In some examples, the parameter component 645 may be configured as or otherwise support a means for selectively modifying one or more operational parameters associated with the second wearable device based at least in part on the first quality metric being greater than the second quality metric.

In some examples, to support selectively modifying the one or more operational parameters, the device state component 660 may be configured as or otherwise support a means for selectively transitioning the second wearable device from an active state to an inactive state, selectively modifying a current provided to one or more sensors of the second wearable device, or both.

In some examples, to support receiving the first physiological data and second physiological data, the physiological data component 625 may be configured as or otherwise support a means for receiving, at the user device from the first wearable device, one or more signals comprising the first physiological data. In some examples, to support receiving the first physiological data and second physiological data, the physiological data component 625 may be configured as or otherwise support a means for receiving, at the user device from the second wearable device, one or more additional signals comprising the second physiological data.

In some examples, to support receiving the first physiological data and second physiological data, the physiological data component 625 may be configured as or otherwise support a means for receiving, at the user device from one of the first wearable device or the second wearable device, one or more signals comprising the first physiological data and the second physiological data.

In some examples, the first physiological data comprises first motion data and the second physiological data comprises second motion data, and the activity determination component 650 may be configured as or otherwise support a means for identifying that the user is engaged in a physical activity based at least in part on the first motion data, the second motion data, or both. In some examples, the first physiological data comprises first motion data and the second physiological data comprises second motion data, and the activity determination component 650 may be configured as or otherwise support a means for classifying the physical activity into a physical activity type selected from a plurality of candidate physical activity types based at least in part on a comparison of the first motion data and the second motion data.

In some examples, the score component 655 may be configured as or otherwise support a means for calculating one or more scores for the user based at least in part on the first physiological data and the second physiological data, the one or more scores comprise a Sleep Score, a Readiness Score, an Activity Score, or any combination thereof

In some examples, the one or more physiological characteristics comprise a metric associated with blood circulation of the user, one or more risk metrics associated with one or more medical conditions, or both. In some examples, the one or more medical conditions comprise Parkinson's, Alzheimer's, stroke, or any combination thereof.

In some examples, the first physiological data comprises a first set of physiological parameters. In some examples, the second physiological data comprises a second set of physiological parameters different from the first set of physiological parameters.

In some examples, the first wearable device, the second wearable device, or both, comprise a wearable ring device.

In some examples, the first wearable device, the second wearable device, or both, collects the first physiological data and the second physiological data, respectively, based on arterial blood flow.

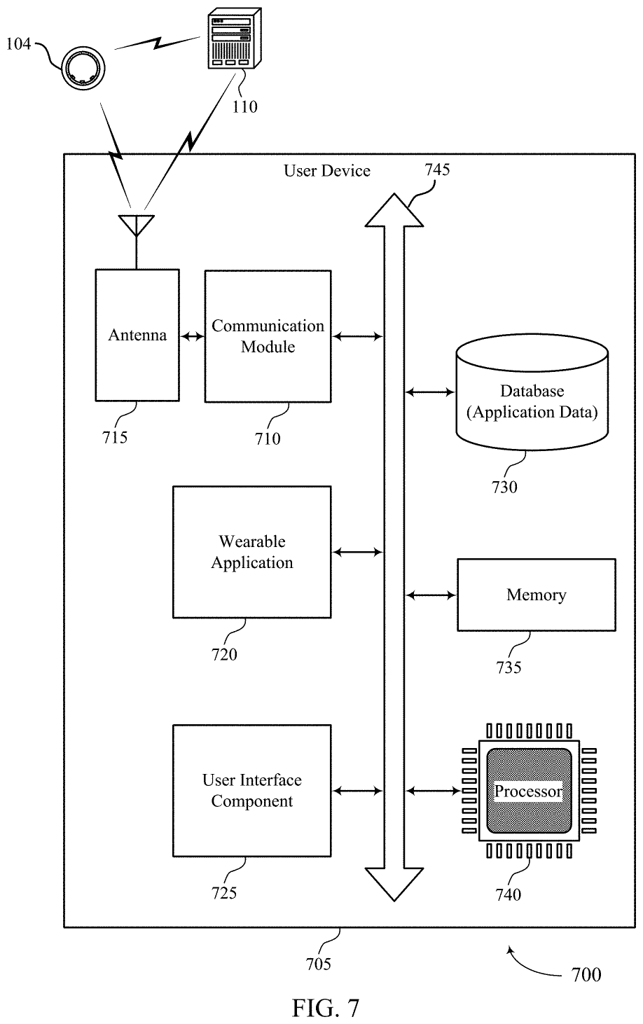

FIG. 7 shows a diagram of a system 700 including a device 705 that supports techniques for multiple wearable devices in accordance with aspects of the present disclosure. The device 705 may be an example of or include the components of a device 505 as described herein. The device 705 may include an example of a user device 106, as described previously herein. The device 705 may include components for bi-directional communications including components for transmitting and receiving communications with a wearable device 104 and a server 110, such as a wearable application 720, a communication module 710, an antenna 715, a user interface component 725, a database (application data) 730, a memory 735, and a processor 740. These components may be in electronic communication or otherwise coupled (e.g., operatively, communicatively, functionally, electronically, electrically) via one or more buses (e.g., a bus 745).

The communication module 710 may manage input and output signals for the device 705 via the antenna 715. The communication module 710 may include an example of the communication module 220-b of the user device 106 shown and described in FIG. 2. In this regard, the communication module 710 may manage communications with the ring 104 and the server 110, as illustrated in FIG. 2. The communication module 710 may also manage peripherals not integrated into the device 705. In some cases, the communication module 710 may represent a physical connection or port to an external peripheral. In some cases, the communication module 710 may utilize an operating system such as iOS®, ANDROID®, MS-DOS®, MS-WINDOWS®, OS/2®, UNIX®, LINUX®, or another known operating system. In other cases, the communication module 710 may represent or interact with a wearable device (e.g., ring 104), modem, a keyboard, a mouse, a touchscreen, or a similar device. In some cases, the communication module 710 may be implemented as part of the processor 740. In some examples, a user may interact with the device 705 via the communication module 710, user interface component 725, or via hardware components controlled by the communication module 710.

In some cases, the device 705 may include a single antenna 715. However, in some other cases, the device 705 may have more than one antenna 715, that may be capable of concurrently transmitting or receiving multiple wireless transmissions. The communication module 710 may communicate bi-directionally, via the one or more antennas 715, wired, or wireless links as described herein. For example, the communication module 710 may represent a wireless transceiver and may communicate bi-directionally with another wireless transceiver. The communication module 710 may also include a modem to modulate the packets, to provide the modulated packets to one or more antennas 715 for transmission, and to demodulate packets received from the one or more antennas 715.

The user interface component 725 may manage data storage and processing in a database 730. In some cases, a user may interact with the user interface component 725. In other cases, the user interface component 725 may operate automatically without user interaction. The database 730 may be an example of a single database, a distributed database, multiple distributed databases, a data store, a data lake, or an emergency backup database.

The memory 735 may include RAM and ROM. The memory 735 may store computer-readable, computer-executable software including instructions that, when executed, cause the processor 740 to perform various functions described herein. In some cases, the memory 735 may contain, among other things, a basic input basic output system (BIOS) that may control basic hardware or software operation such as the interaction with peripheral components or devices.

The processor 740 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 740 may be configured to operate a memory array using a memory controller. In other cases, a memory controller may be integrated into the processor 740. The processor 740 may be configured to execute computer-readable instructions stored in a memory 735 to perform various functions (e.g., functions or tasks supporting a method and system for sleep staging algorithms).

The wearable application 720 may support collecting physiological data from a plurality of wearable devices in accordance with examples as disclosed herein. For example, the wearable application 720 may be configured as or otherwise support a means for receiving first physiological data associated with a user from a first wearable device worn at a first position on the user. The wearable application 720 may be configured as or otherwise support a means for receiving second physiological data associated with the user from a second wearable device worn at a second position on the user different from the first position. The wearable application 720 may be configured as or otherwise support a means for determining one or more physiological characteristics associated with the user based at least in part on a comparison of the first physiological data and the second physiological data. The wearable application 720 may be configured as or otherwise support a means for causing a graphical user interface of a user device to display an indication of the one or more physiological characteristics.

By including or configuring the wearable application 720 in accordance with examples as described herein, the device 705 may support techniques for multiple wearable devices that may result in improved data collection for a user, improved alerts or instructions provided to a user, improved user experience related to multiple devices, improved coordination between devices, reduced power consumption, and improve battery life.

The wearable application 720 may include an application (e.g., “app”), program, software, or other component that is configured to facilitate communications with a ring 104, server 110, other user devices 106, and the like. For example, the wearable application 720 may include an application executable on a user device 106 that is configured to receive data (e.g., physiological data) from a ring 104, perform processing operations on the received data, transmit and receive data with the servers 110, and cause presentation of data to a user 102.

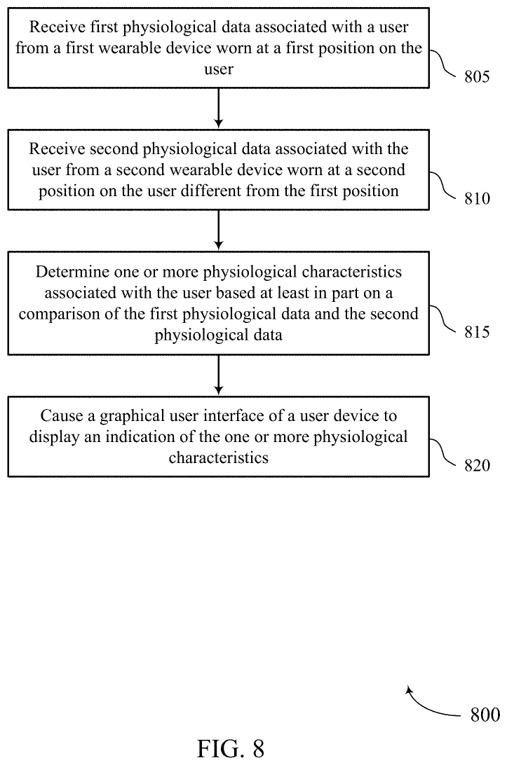

FIG. 8 shows a flowchart illustrating a method 800 that supports techniques for multiple wearable devices in accordance with aspects of the present disclosure. The operations of the method 800 may be implemented by a user device or its components as described herein. For example, the operations of the method 800 may be performed by a user device as described with reference to FIGS. 1 through 7. In some examples, a user device may execute a set of instructions to control the functional elements of the user device to perform the described functions. Additionally or alternatively, the user device may perform aspects of the described functions using special-purpose hardware.

At 805, the method may include receiving first physiological data associated with a user from a first wearable device worn at a first position on the user. The operations of 805 may be performed in accordance with examples as disclosed herein. In some examples, aspects of the operations of 805 may be performed by a physiological data component 625 as described with reference to FIG. 6.

At 810, the method may include receiving second physiological data associated with the user from a second wearable device worn at a second position on the user different from the first position. The operations of 810 may be performed in accordance with examples as disclosed herein. In some examples, aspects of the operations of 810 may be performed by a physiological data component 625 as described with reference to FIG. 6.

At 815, the method may include determining one or more physiological characteristics associated with the user based at least in part on a comparison of the first physiological data and the second physiological data. The operations of 815 may be performed in accordance with examples as disclosed herein. In some examples, aspects of the operations of 815 may be performed by a physiological data comparison component 630 as described with reference to FIG. 6.

At 820, the method may include causing a graphical user interface of a user device to display an indication of the one or more physiological characteristics. The operations of 820 may be performed in accordance with examples as disclosed herein. In some examples, aspects of the operations of 820 may be performed by a data display component 635 as described with reference to FIG. 6.

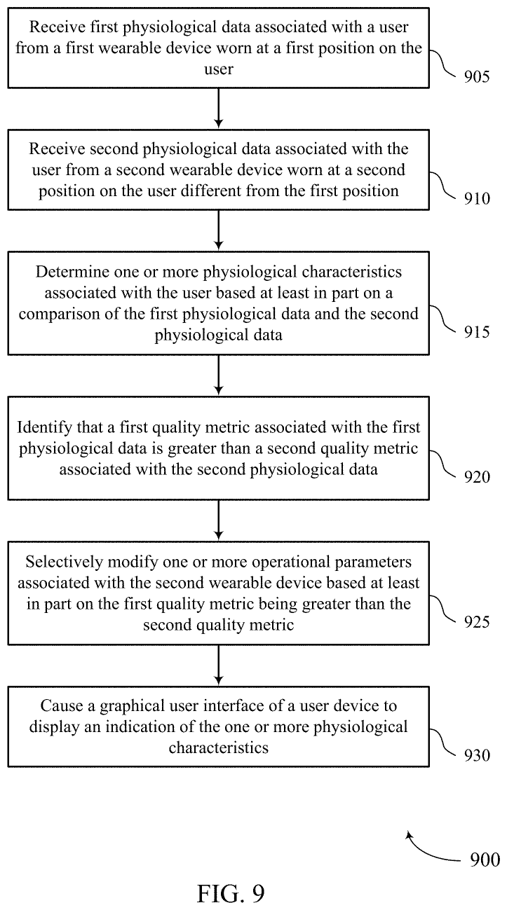

FIG. 9 shows a flowchart illustrating a method 900 that supports techniques for multiple wearable devices in accordance with aspects of the present disclosure. The operations of the method 900 may be implemented by a user device or its components as described herein. For example, the operations of the method 900 may be performed by a user device as described with reference to FIGS. 1 through 7. In some examples, a user device may execute a set of instructions to control the functional elements of the user device to perform the described functions. Additionally or alternatively, the user device may perform aspects of the described functions using special-purpose hardware.

At 905, the method may include receiving first physiological data associated with a user from a first wearable device worn at a first position on the user. The operations of 905 may be performed in accordance with examples as disclosed herein. In some examples, aspects of the operations of 905 may be performed by a physiological data component 625 as described with reference to FIG. 6.

At 910, the method may include receiving second physiological data associated with the user from a second wearable device worn at a second position on the user different from the first position. The operations of 910 may be performed in accordance with examples as disclosed herein. In some examples, aspects of the operations of 910 may be performed by a physiological data component 625 as described with reference to FIG. 6.

At 915, the method may include determining one or more physiological characteristics associated with the user based at least in part on a comparison of the first physiological data and the second physiological data. The operations of 915 may be performed in accordance with examples as disclosed herein. In some examples, aspects of the operations of 915 may be performed by a physiological data comparison component 630 as described with reference to FIG. 6.

At 920, the method may include identifying that a first quality metric associated with the first physiological data is greater than a second quality metric associated with the second physiological data. The operations of 920 may be performed in accordance with examples as disclosed herein. In some examples, aspects of the operations of 920 may be performed by a quality metric component 640 as described with reference to FIG. 6.

At 925, the method may include selectively modifying one or more operational parameters associated with the second wearable device based at least in part on the first quality metric being greater than the second quality metric. The operations of 925 may be performed in accordance with examples as disclosed herein. In some examples, aspects of the operations of 925 may be performed by a parameter component 645 as described with reference to FIG. 6.

At 930, the method may include causing a graphical user interface of a user device to display an indication of the one or more physiological characteristics. The operations of 930 may be performed in accordance with examples as disclosed herein. In some examples, aspects of the operations of 930 may be performed by a data display component 635 as described with reference to FIG. 6.

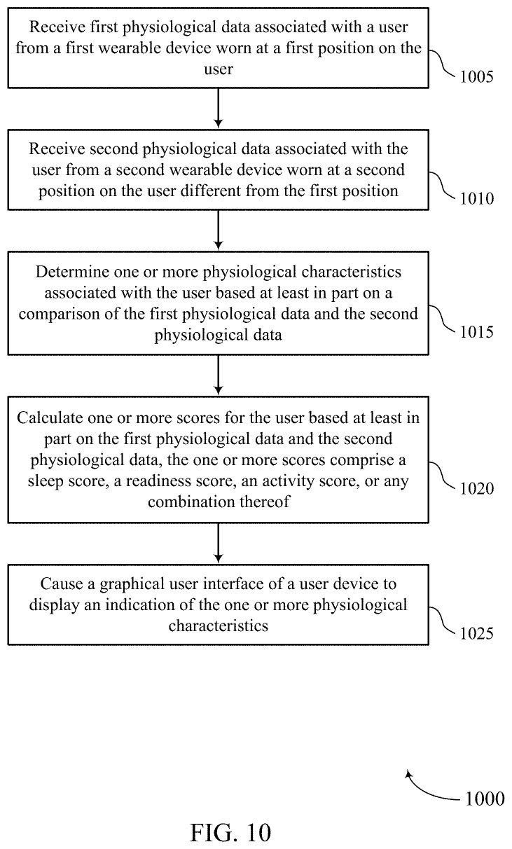

FIG. 10 shows a flowchart illustrating a method 1000 that supports techniques for multiple wearable devices in accordance with aspects of the present disclosure. The operations of the method 1000 may be implemented by a user device or its components as described herein. For example, the operations of the method 1000 may be performed by a user device as described with reference to FIGS. 1 through 7. In some examples, a user device may execute a set of instructions to control the functional elements of the user device to perform the described functions. Additionally or alternatively, the user device may perform aspects of the described functions using special-purpose hardware.

At 1005, the method may include receiving first physiological data associated with a user from a first wearable device worn at a first position on the user. The operations of 1005 may be performed in accordance with examples as disclosed herein. In some examples, aspects of the operations of 1005 may be performed by a physiological data component 625 as described with reference to FIG. 6.

At 1010, the method may include receiving second physiological data associated with the user from a second wearable device worn at a second position on the user different from the first position. The operations of 1010 may be performed in accordance with examples as disclosed herein. In some examples, aspects of the operations of 1010 may be performed by a physiological data component 625 as described with reference to FIG. 6.

At 1015, the method may include determining one or more physiological characteristics associated with the user based at least in part on a comparison of the first physiological data and the second physiological data. The operations of 1015 may be performed in accordance with examples as disclosed herein. In some examples, aspects of the operations of 1015 may be performed by a physiological data comparison component 630 as described with reference to FIG. 6.

At 1020, the method may include calculating one or more scores for the user based at least in part on the first physiological data and the second physiological data, the one or more scores comprise a Sleep Score, a Readiness Score, an Activity Score, or any combination thereof. The operations of 1020 may be performed in accordance with examples as disclosed herein. In some examples, aspects of the operations of 1020 may be performed by a score component 655 as described with reference to FIG. 6.

At 1025, the method may include causing a graphical user interface of a user device to display an indication of the one or more physiological characteristics. The operations of 1025 may be performed in accordance with examples as disclosed herein. In some examples, aspects of the operations of 1025 may be performed by a data display component 635 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method for collecting physiological data from a plurality of wearable devices is described. The method may include receiving first physiological data associated with a user from a first wearable device worn at a first position on the user, receiving second physiological data associated with the user from a second wearable device worn at a second position on the user different from the first position, determining one or more physiological characteristics associated with the user based at least in part on a comparison of the first physiological data and the second physiological data, and causing a GUI of a user device to display an indication of the one or more physiological characteristics.

An apparatus for collecting physiological data from a plurality of wearable devices is described. The apparatus may include a processor, memory coupled with the processor, and instructions stored in the memory. The instructions may be executable by the processor to cause the apparatus to receive first physiological data associated with a user from a first wearable device worn at a first position on the user, receive second physiological data associated with the user from a second wearable device worn at a second position on the user different from the first position, determine one or more physiological characteristics associated with the user based at least in part on a comparison of the first physiological data and the second physiological data, and cause a GUI of a user device to display an indication of the one or more physiological characteristics.

Another apparatus for collecting physiological data from a plurality of wearable devices is described. The apparatus may include means for receiving first physiological data associated with a user from a first wearable device worn at a first position on the user, means for receiving second physiological data associated with the user from a second wearable device worn at a second position on the user different from the first position, means for determining one or more physiological characteristics associated with the user based at least in part on a comparison of the first physiological data and the second physiological data, and means for causing a GUI of a user device to display an indication of the one or more physiological characteristics.

A non-transitory computer-readable medium storing code for collecting physiological data from a plurality of wearable devices is described. The code may include instructions executable by a processor to receive first physiological data associated with a user from a first wearable device worn at a first position on the user, receive second physiological data associated with the user from a second wearable device worn at a second position on the user different from the first position, determine one or more physiological characteristics associated with the user based at least in part on a comparison of the first physiological data and the second physiological data, and cause a GUI of a user device to display an indication of the one or more physiological characteristics.

Some examples of the method, apparatuses, and non-transitory computer-readable medium described herein may further include operations, features, means, or instructions for identifying that a first quality metric associated with the first physiological data may be greater than a second quality metric associated with the second physiological data and selectively modifying one or more operational parameters associated with the second wearable device based at least in part on the first quality metric being greater than the second quality metric.

In some examples of the method, apparatuses, and non-transitory computer-readable medium described herein, selectively modifying the one or more operational parameters may include operations, features, means, or instructions for selectively transitioning the second wearable device from an active state to an inactive state, selectively modifying a current provided to one or more sensors of the second wearable device, or both.

In some examples of the method, apparatuses, and non-transitory computer-readable medium described herein, receiving the first physiological data and second physiological data may include operations, features, means, or instructions for receiving, at the user device from the first wearable device, one or more signals comprising the first physiological data and receiving, at the user device from the second wearable device, one or more additional signals comprising the second physiological data.

In some examples of the method, apparatuses, and non-transitory computer-readable medium described herein, receiving the first physiological data and second physiological data may include operations, features, means, or instructions for receiving, at the user device from one of the first wearable device or the second wearable device, one or more signals comprising the first physiological data and the second physiological data.

In some examples of the method, apparatuses, and non-transitory computer-readable medium described herein, the first physiological data comprises first motion data and the second physiological data comprises second motion data and the method, apparatuses, and non-transitory computer-readable medium may include further operations, features, means, or instructions for identifying that the user may be engaged in a physical activity based at least in part on the first motion data, the second motion data, or both and classifying the physical activity into a physical activity type selected from a plurality of candidate physical activity types based at least in part on a comparison of the first motion data and the second motion data.

Some examples of the method, apparatuses, and non-transitory computer-readable medium described herein may further include operations, features, means, or instructions for calculating one or more scores for the user based at least in part on the first physiological data and the second physiological data, the one or more scores comprise a Sleep Score, a Readiness Score, an Activity Score, or any combination thereof.

In some examples of the method, apparatuses, and non-transitory computer-readable medium described herein, the one or more physiological characteristics comprise a metric associated with blood circulation of the user, one or more risk metrics associated with one or more medical conditions, or both and the one or more medical conditions comprise Parkinson's, Alzheimer's, stroke, or any combination thereof

In some examples of the method, apparatuses, and non-transitory computer-readable medium described herein, the first physiological data comprises a first set of physiological parameters and the second physiological data comprises a second set of physiological parameters different from the first set of physiological parameters.

In some examples of the method, apparatuses, and non-transitory computer-readable medium described herein, the first wearable device, the second wearable device, or both, comprise a wearable ring device.

In some examples of the method, apparatuses, and non-transitory computer-readable medium described herein, the first wearable device, the second wearable device, or both, collects the first physiological data and the second physiological data, respectively, based on arterial blood flow.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for collecting physiological data from a plurality of wearable devices, comprising:
receiving first physiological data associated with a user from a first wearable device worn at a first position on the user;
receiving second physiological data associated with the user from a second wearable device worn at a second position on the user different from the first position;
determining one or more physiological characteristics associated with the user based at least in part on a comparison of the first physiological data and the second physiological data; and
causing a graphical user interface of a user device to display an indication of the one or more physiological characteristics.

2. The method of claim 1, further comprising:
identifying that a first quality metric associated with the first physiological data is greater than a second quality metric associated with the second physiological data; and
selectively modifying one or more operational parameters associated with the second wearable device based at least in part on the first quality metric being greater than the second quality metric.

3. The method of claim 2, wherein selectively modifying the one or more operational parameters comprises:
selectively transitioning the second wearable device from an active state to an inactive state, selectively modifying a current provided to one or more sensors of the second wearable device, or both.

4. The method of claim 1, wherein receiving the first physiological data and second physiological data comprises:
receiving, at the user device from the first wearable device, one or more signals comprising the first physiological data; and
receiving, at the user device from the second wearable device, one or more additional signals comprising the second physiological data.

5. The method of claim 1, wherein receiving the first physiological data and second physiological data comprises:
receiving, at the user device from one of the first wearable device or the second wearable device, one or more signals comprising the first physiological data and the second physiological data.

6. The method of claim 1, wherein the first physiological data comprises first motion data and the second physiological data comprises second motion data, the method further comprising:
identifying that the user is engaged in a physical activity based at least in part on the first motion data, the second motion data, or both; and
classifying the physical activity into a physical activity type selected from a plurality of candidate physical activity types based at least in part on a comparison of the first motion data and the second motion data.

7. The method of claim 1, further comprising:
calculating one or more scores for the user based at least in part on the first physiological data and the second physiological data, the one or more scores comprising a sleep score, a readiness score, an activity score, or any combination thereof

8. The method of claim 7, wherein the one or more physiological characteristics comprise a metric associated with blood circulation of the user, one or more risk metrics associated with one or more medical conditions, or both, and wherein the one or more medical conditions comprise Parkinson's, Alzheimer's, stroke, or any combination thereof

9. The method of claim 1, wherein the first physiological data comprises a first set of physiological parameters, and wherein the second physiological data comprises a second set of physiological parameters different from the first set of physiological parameters.

10. The method of claim 1, wherein the first wearable device, the second wearable device, or both, comprise a wearable ring device.

11. The method of claim 1, wherein the first wearable device, the second wearable device, or both, collects the first physiological data and the second physiological data, respectively, based on arterial blood flow.

12. An apparatus for collecting physiological data from a plurality of wearable devices, comprising:
a processor;
memory coupled with the processor; and
instructions stored in the memory and executable by the processor to cause the apparatus to:
receive first physiological data associated with a user from a first wearable device worn at a first position on the user;
receive second physiological data associated with the user from a second wearable device worn at a second position on the user different from the first position;
determine one or more physiological characteristics associated with the user based at least in part on a comparison of the first physiological data and the second physiological data; and
cause a graphical user interface of a user device to display an indication of the one or more physiological characteristics.

13. The apparatus of claim 12, wherein the instructions are further executable by the processor to cause the apparatus to:
identify that a first quality metric associated with the first physiological data is greater than a second quality metric associated with the second physiological data; and
selectively modify one or more operational parameters associated with the second wearable device based at least in part on the first quality metric being greater than the second quality metric.

14. The apparatus of claim 13, wherein the instructions to selectively modify the one or more operational parameters are executable by the processor to cause the apparatus to:
selectively transition the second wearable device from an active state to an inactive state, selectively modify a current provided to one or more sensors of the second wearable device, or both.

15. The apparatus of claim 12, wherein the instructions to receive the first physiological data and second physiological data are executable by the processor to cause the apparatus to:
receive, at the user device from the first wearable device, one or more signals comprising the first physiological data; and
receive, at the user device from the second wearable device, one or more additional signals comprising the second physiological data.

16. The apparatus of claim 12, wherein the instructions to receive the first physiological data and second physiological data are executable by the processor to cause the apparatus to:
receive, at the user device from one of the first wearable device or the second wearable device, one or more signals comprising the first physiological data and the second physiological data.

17. The apparatus of claim 12, wherein the first physiological data comprises first motion data and the second physiological data comprises second motion data, and the instructions are further executable by the processor to cause the apparatus to:
identify that the user is engaged in a physical activity based at least in part on the first motion data, the second motion data, or both; and
classify the physical activity into a physical activity type selected from a plurality of candidate physical activity types based at least in part on a comparison of the first motion data and the second motion data.

18. The apparatus of claim 12, wherein the instructions are further executable by the processor to cause the apparatus to:
calculate one or more scores for the user based at least in part on the first physiological data and the second physiological data, the one or more scores comprise a sleep score, a readiness score, an activity score, or any combination thereof.

19. The apparatus of claim 12, wherein the first physiological data comprises a first set of physiological parameters, and the second physiological data comprises a second set of physiological parameters different from the first set of physiological parameters.

20. A non-transitory computer-readable medium storing code for collecting physiological data from a plurality of wearable devices, the code comprising instructions executable by a processor to:
receive first physiological data associated with a user from a first wearable device worn at a first position on the user;
receive second physiological data associated with the user from a second wearable device worn at a second position on the user different from the first position;
determine one or more physiological characteristics associated with the user based at least in part on a comparison of the first physiological data and the second physiological data; and
cause a graphical user interface of a user device to display an indication of the one or more physiological characteristics.
